(19) United States
(12) Patent Application Publication
(10) Pub. No.: US 2026/0108284 A1
(43) Pub. Date: Apr. 23, 2026

(54) Compliant Pre-Tensioned Implant for Patella Fractures

(71) Applicant: The Penn State Research Foundation, University Park, PA (US)

(72) Inventors: Connor Huxman, University Park, PA (US); Gary Updegrove, Hershey, PA (US); April Armstrong, Hershey, PA (US); Gregory Lewis, Hershey, PA (US); Jared Butler, University Park, PA (US)

(73) Assignee: The Penn State Research Foundation, University Park, PA (US)

(21) Appl. No.: 19/475,524

(22) PCT Filed: Dec. 17, 2024
(86) PCT No.: PCT/US2024/060474

§ 371 Date: Oct. 15, 2025

Related U.S. Application Data
(60) Provisional application No. 63/611,310, filed on Dec. 18, 2023.

(51) Int. Cl. A61B 17/80

(52) U.S. Cl. CPC A61B 17/8004; A61B 17/8023; A61B 17/8061; A61B 17/809

(57) ABSTRACT

A patella reinforcing member is disclosed. The patella reinforcing member is configured as a compliant mechanism that may be pre-tensioned/stretched to fit around a fractured patella. The member may then be anchored to the patella—for example, via fasteners and/or hooks—and the pre-tensioning forces may be released as the member returns to its initial, non-tensioned position. The member may thereby deliver targeted anterior compression between fractured bone fragments.

### CROSS-REFERENCE TO RELATED APPLICATIONS

This patent application is related to and claims the benefit of priority of U.S. Provisional Application 63/611,310, filed on Dec. 18, 2023, the entire contents of which is incorporated by reference.

### FIELD

The present disclosure generally relates to devices and methods configured to repair patella fractures. Specifically, an orthopedic reinforcing member that leverages pre-tensioning such that the member may be pre-tensioned/stretched to fit around a fractured patella is disclosed. Pre-tension forces may be released as the member attempts to return to its resting position, thus delivering targeted compression between fractured bone fragments.

### BACKGROUND

Patella (e.g., the kneecap) fractures have remained challenging to treat and have a high rate of reoperation, implant failure, and/or patient discomfort. These challenges are due in part to the very large tensile forces experienced by the patella during leg extension (e.g., pulling upwards through the quadriceps tendon and downwards through the patella tendons). Surgical techniques designed to heal patella fractures exist and commonly use implant plates together with lag screws and optional wiring. However, existing techniques are often overly rigid and have been found to cause patient discomfort, irritate soft tissue, and/or cause loosened wires under large tensile forces, and may actually inhibit bone healing. Compression between bone fragments is known to promote biomechanical stability and primary bone healing, but compression is not obtained with some current techniques.

Comminuted fractures (e.g., fractures containing more than two bone fragments) have been found to be especially difficult to treat. Thirty to fifty percent of patients with treated comminuted fractures suffer from persistent anterior knee pain, and fifteen to thirty percent of patients suffer from functional impairment. Moreover, the hardware removal rate in the first year following use of lag screws and/or wiring to treat comminuted patella fractures is ten percent.

### SUMMARY

We have determined that there is a need to develop a flexible patella implant that may provide stability to a fracture and compression between bone fragments, while also accommodating tensile loading of the patella. We have therefore developed a patella reinforcing member configured as a compliant mechanism that may achieve movement and force transmission through elastic deformation (e.g., stretching) of flexible components. In particular, the member may be pre-tensioned/stretched to fit around a fractured patella. The member may then be anchored to the patella—for example, via fasteners and/or hooks—and the pre-tensioning forces may be released as the member returns to its initial, non-tensioned position. The member may thereby stabilize and deliver targeted compression between fractured bone fragments.

The patella reinforcing member may have various topologies and configurations to deliver single plane (e.g., linear) and/or multi-plane (e.g., radial) compression to bone fragments. For example, a transverse fracture including two bone fragments may benefit from single plane, linear compression, while a comminuted fracture including more than two bone fragments may benefit multi-plane compression from more than one direction.

In an exemplary embodiment, patella reinforcing member comprises a body having a first portion, a second portion opposite the first portion, and an intermediate portion between the first portion and the second portion; the intermediate portion sized and configured to be resiliently moveable from an initial position to a plurality of different stretched positions, the intermediate portion configured to be resiliently biased to return to the initial position when in any of the plurality of different stretched positions; the first portion of the body being attachable to a first side of a patella bone; the second portion of the body being attachable to a second side of the patella bone opposite the first side of the patella bone such that the intermediate portion is in at least one of the plurality of different stretched positions while the first portion of the body is attached to the first side of the patella bone and the second portion of the body is attached to the second side of the patella bone such that the intermediate portion exerts a compressive force on the patella bone.

In some embodiments, the first portion of the body is attachable to the first side of the patella bone via one or more first fasteners and the second portion of the body is attachable to the second side of the patella bone via one or more second fasteners.

In some embodiments, the first portion of the body is attachable to the first side of the patella bone via one or more first fasteners and the second portion of the body is attachable to the second side of the patella bone via the one or more first fasteners.

In some embodiments, the one or more first fasteners are comprised of a first screw or bolt and a second screw or bolt.

In some embodiments, the intermediate portion is sized and configured to have an array of openings defined therein, the array of openings being a honeycomb pattern of openings that are each a first size when the intermediate portion is in the initial position and are each a second larger size when the intermediate portion is in one or more of the stretched positions.

In some embodiments, the intermediate portion is sized and configured to have an array of openings defined therein, the array of openings being polygonal shaped openings that are a first size when the intermediate portion is in the initial position and are a second larger size when the intermediate portion is in one or more of the stretched positions.

In some embodiments, the intermediate portion has one or more auxetic structures; one or more serpentine flexure regions; lattice structure; a topology-geometric profile that facilitates linear motion; and/or one or more re-entrant honeycomb structures.

In some embodiments, the intermediate portion has a first segment defining a first opening adjacent the first side that has a first size when the intermediate portion is in the initial position and has second larger sizes when the intermediate portion is in the stretched positions; a second segment defining a second opening adjacent the second side that has a first size when the intermediate portion is in the initial position and has second larger sizes when the intermediate portion is in the stretched positions.

In some embodiments, the intermediate portion has a third segment defining a third opening between the first segment and the second segment, the third opening being defined such that the third opening has a first size when the intermediate portion is in the initial position and has second larger sizes when the intermediate portion is in the stretched positions.

In some embodiments, the intermediate portion has a fourth segment defining a fourth opening between the first segment and the third segment, the fourth opening being defined such that the fourth opening has a first size when the intermediate portion is in the initial position and has second larger sizes when the intermediate portion is in the stretched positions; and a fifth segment defining a fifth opening between the second segment and the third segment, the fifth opening being defined such that the fifth opening has a first size when the intermediate portion is in the initial position and has second larger sizes when the intermediate portion is in the stretched positions.

In some embodiments, the first portion of the body has one or more projections configured to hook onto the first side of the patella bone.

In some embodiments, the first portion of the body has a plurality of spaced apart holes to receive first fasteners therein for attaching the first portion of the body to the first side of the patella bone.

In some embodiments, the second portion of the body has one or more projections configured to hook onto the second side of the patella bone.

In an exemplary embodiment, a method of attaching a patella reinforcing member onto a patella bone that is fractured comprises positioning the patella reinforcing member, as described herein, onto the patella bone so that the intermediate portion is in at least one of the plurality of different stretched positions while the first portion of the body is attached to the first side of the patella bone and the second portion of the body is attached to the second side of the patella bone such that the intermediate portion exerts a compressive force on the patella bone.

In some embodiments, the method comprises cutting into a knee of a patient to access the patella bone that is fractured.

In some embodiments, the method comprises passing fasteners through the patella reinforcing member and the patella bone to attach the patella reinforcing member to the patella bone so that the intermediate portion is in at least one of the plurality of different stretched positions while the first portion of the body is attached to the first side of the patella bone and the second portion of the body is attached to the second side of the patella bone such that the intermediate portion exerts a compressive force on the patella bone.

In an exemplary embodiment, a patella reinforcing member comprises a body having a first side portion, a second side portion opposite the first side portion, and a third portion between and connecting the first side portion and the second side portion; a first channel extending from an edge of the body and between the first side portion and the second side portion, a second channel extending between the first side portion and the third portion, and a third channel extending between second side portion and the third portion, wherein the body is sized and configured to be resiliently moveable from an initial position to a plurality of different stretched positions, the body configured to be resiliently biased to return to the initial position when in any of the plurality of different stretched positions, and wherein the first side portion is attachable to a first side of a patella bone, the second side portion is attachable to a second side of the patella bone, and the third portion is attachable to a distal side of the patella bone, such that when the body is in at least one of the plurality of different stretched positions when the first side portion is attached to the first side of the patella bone, the second side portion is attached to the second side of the patella bone, and the third portion is attached to the distal side of the patella bone, the body exerts a radial compressive force on the patella bone.

In some embodiments, the first side portion of the body is attachable to the first side of the patella bone via one or more first fasteners, the second side portion of the body is attachable to the second side of the patella bone via one or more second fasteners, and the third portion of the body is attachable to the distal side of the patella bone via one or more third fasteners.

In some embodiments, the first side portion of the body has one or more projections configured to hook onto the first side of the patella bone.

In some embodiments, the first side portion of the body has a plurality of spaced apart holes to receive first fasteners therein for attaching the first side portion of the body to the first side of the patella bone.

In some embodiments, the second side portion of the body has one or more projections configured to hook onto the second side of the patella bone.

In some embodiments, the second side portion of the body has a plurality of spaced apart holes to receive second fasteners therein for attaching the second side portion of the body to the second side of the patella bone.

In some embodiments, the third portion of the body has one or more projections configured to hook onto the distal side of the patella bone.

In some embodiments, the third portion of the body has a plurality of spaced apart holes to receive third fasteners therein for attaching the third portion of the body to the distal side of the patella bone.

In an exemplary embodiment, a method of attaching a patella reinforcing member onto a patella bone that is fractured comprises positioning the patella reinforcing member, as described herein, onto the patella bone so that the body is in at least one of the plurality of different stretched positions while the first side portion of the body is attached to the first side of the patella bone, the second side portion of the body is attached to the second side of the patella bone, and the third portion is attached to the distal side of the patella bone such that the body exerts a radial compressive force on the patella bone.

In some embodiments, the method comprises cutting into a knee of a patient to access the patella bone that is fractured.

In some embodiments, the method comprises passing fasteners through the patella reinforcing member and the patella bone to attach the patella reinforcing member to the patella bone so that the body is in at least one of the plurality of different stretched positions while the first side portion of the body is attached to the first side of the patella bone, the second side portion of the body is attached to the second side of the patella bone, and the third portion is attached to the distal side of the patella bone such that the body exerts a radial compressive force on the patella bone.

In an exemplary embodiment, a patella reinforcing member comprises a body having a first portion, a second portion, and a third portion arranged annularly; an intermediate portion positioned between the first portion, the second portion, and the third portion, the intermediate portion sized and configured to be resiliently moveable from an initial position to a plurality of different stretched positions, the intermediate portion configured to be resiliently biased to return to the initial position when in any of the plurality of different stretched positions; the first portion of the body being attachable to a first side of a patella bone, the second portion of the body being attachable to a second side of a patella bone, and the third portion of the body being attachable to a third side of a patella bone, such that when the body is in at least one of the plurality of different stretched positions when the first portion is attached to the first side of the patella bone, the second portion is attached to the second side of the patella bone, and the third portion is attached to the third side of the patella bone, the body exerts a radial compressive force on the patella bone.

In some embodiments, the first portion of the body is attachable to the first side of the patella bone via one or more first fasteners, the second portion of the body is attachable to the second side of the patella bone via one or more second fasteners, and the third portion of the body is attachable to the third side of the patella bone via one or more third fasteners.

In some embodiments, the intermediate portion is sized and configured to have a flexible geometry with one or more spiral flexures, the spiral flexures are each a first size when the intermediate portion is in the initial position and are each a second larger size when the intermediate portion is in one or more of the stretched positions.

In some embodiments, the first portion of the body has one or more projections configured to hook onto the first side of the patella bone.

In some embodiments, the first portion of the body has a plurality of spaced apart holes to receive first fasteners therein for attaching the first portion of the body to the first side of the patella bone.

In some embodiments, the second portion of the body has one or more projections configured to hook onto the second side of the patella bone.

In some embodiments, the second portion of the body has a plurality of spaced apart holes to receive second fasteners therein for attaching the second portion of the body to the second side of the patella bone.

In some embodiments, the third portion of the body has one or more projections configured to hook onto the third side of the patella bone.

In some embodiments, the third portion of the body has a plurality of spaced apart holes to receive third fasteners therein for attaching the third portion of the body to the distal side of the patella bone.

In an exemplary embodiment, a method of attaching a patella reinforcing member onto a patella bone that is fractured comprises positioning the patella reinforcing member, as described herein, onto the patella bone so that the body is in at least one of the plurality of different stretched positions while the first portion of the body is attached the first side of the patella bone, the second portion of the body is attached to the second side of the patella bone, and the third portion is attached to the third side of the patella bone such that the body exerts a radial compressive force on the patella bone.

In some embodiments, the method comprises cutting into a knee of a patient to access the patella bone that is fractured.

In some embodiments, the method comprises passing fasteners through the patella reinforcing member and the patella bone to attach the patella reinforcing member to the patella bone so that the body is in at least one of the plurality of different stretched positions while the first portion of the body is attached to the first side of the patella bone, the second portion of the body is attached to the second side of the patella bone, and the third portion is attached to the third side of the patella bone such that the body exerts a radial compressive force on the patella bone.

### BRIEF DESCRIPTION OF THE DRAWINGS

The above and other objects, aspects, features, advantages, and possible applications of embodiments of the present innovation will be more apparent from the following more particular description thereof, presented in conjunction with the following drawings. Like reference numbers used in the drawings may identify like components.

FIG. 1 is a schematic illustration of an exemplary knee joint of a patient.

FIG. 2 is a schematic illustration of the anterior surface of an exemplary patella (e.g., kneecap) of patient.

FIG. 3 is a perspective view of an exemplary patella reinforcing member configured for linear compression of patella bone fragments.

FIG. 4 is a perspective view of an exemplary patella reinforcing member configured for linear compression of patella bone fragments. The member is anchored to the patella with fasteners and hooks.

FIG. 5A is a front view of an exemplary patella reinforcing member configured for linear compression of patella bone fragments and including an exemplary array of openings.

FIG. 5B is a side view of the exemplary patella reinforcing member shown in FIG. 5A.

FIG. 5C is a perspective view of the exemplary patella reinforcing member shown in FIG. 5A.

FIG. 5D is a perspective view of the exemplary patella reinforcing member shown in FIG. 5A.

FIG. 6A is a front view of the exemplary patella reinforcing member configured for linear compression of patella bone fragments and including an exemplary array of openings.

FIG. 6B is a side view of the exemplary patella reinforcing member shown in FIG. 6A.

FIG. 6C is a perspective view of the exemplary patella reinforcing member shown in FIG. 6A.

FIG. 6D is a perspective view of the exemplary patella reinforcing member shown in FIG. 6A.

FIG. 7 is a flow chart illustrating an exemplary process for utilization of an exemplary embodiment of the patella reinforcing member configured for linear compression of patella bone fragments in which the patella reinforcing member is originally provided in its initial position and is subsequently attached to a patella in a stretched position so that the patella reinforcing member can exert a compressing force while anchored onto the patella via resilient motion that is biased toward moving the patella reinforcing member back to its initial position. The exemplary embodiment of the patella reinforcing member includes an exemplary array of openings.

FIG. 8 is a schematic illustration of a flow chart illustrating an exemplary process for utilization of an exemplary embodiment of the patella reinforcing member configured for linear compression of patella bone fragments in its initial position and subsequently attached to a patella in a stretched position so that it can exert a compressive force on the patella due to being biased to move from its stretched position back to its initial position while being anchored onto the patella. The exemplary embodiment of the patella reinforcing member includes exemplary flexure elements.

FIG. 9 is a schematic illustration of a flow chart of an exemplary embodiment of a process for utilization of an exemplary embodiment of the patella reinforcing member configured for multi-plane/radial compression of patella bone fragments in which the patella reinforcing member is initially in its initial position and is subsequently moved to a stretched position for being attached to a patella so that the patella reinforcing member can exert a compression force on the patella while it is anchored onto the patella via resilient biased motion of the patella reinforcing member biasing the patella reinforcing member from its stretched position back to its original initial position.

FIG. 10 is a perspective view an exemplary patella reinforcing member configured for multi-plane/radial compression of patella bone fragments.

FIG. 11 is a schematic flow chart illustrating an exemplary method of using a patella reinforcing member to promote healing of patella bone fragments.

FIG. 12 includes results from conducted computational biomechanics simulations comparing standard lag screw fixation to patella reinforcing members, demonstrating that the patella reinforcing members can increase total interfragmentary compression and concentrate compression anteriorly, where fracture dislocation is prone to occur.

### DETAILED DESCRIPTION

The following description is of exemplary embodiments and methods of use that are presently contemplated for carrying out the present invention. This description is not to be taken in a limiting sense, but is made merely for the purpose of describing the general principles and features of various aspects of the present invention. The scope of the present invention is not limited by this description.

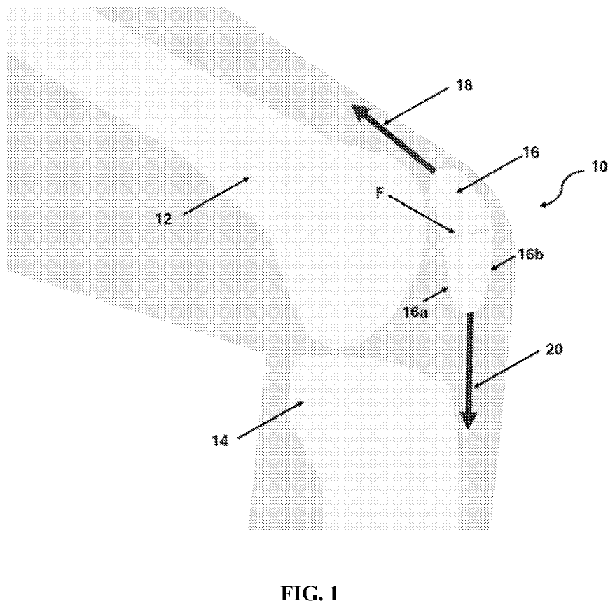

FIG. 1 illustrates a typical knee joint 10 of a patient. The knee joint 10 is formed between the lower end of the femur 12 and the upper end of the tibia 14. The patella (e.g., the kneecap) 16 is small, disc-shaped bone that is movable over the lower end of the femur 12. The patella 16 has a posterior, joint-facing surface 16a that is shaped and configured to engage the femur 12, and an anterior, outward facing surface 16b that is typically, but not necessarily, convex/rounded. The patella 16 is a sesamoid bone, meaning that it is embedded within one or more tendons. In particular, the patella 16 is connected to the quadriceps tendon 18 and the patella tendon 20. The quadriceps tendon 18 is further connected to the quadriceps muscle, which is attached to the femur 12, and the patella tendon 20 is further connected to the tibia 14. The patella 16 may experience large tensile loading as a patient actively bends and extends their leg, pulled upward by the quadriceps tendon 18 and/or downwards by the patella tendon 20 during activities. The patella 16 simultaneously experiences anterior directed loading from the femur 12, especially with the knee flexed.

A patella fracture F refers to a break in the patella 16 and can vary in type and severity. Exemplary fractures may include, but are not limited to, a transverse fracture and a comminuted fracture. A “transverse fracture” runs horizontally across the patella (see e.g., FIG. 1) and usually includes breakage into two bone fragments, while a “comminuted fracture” includes breakage into more than two bone fragments. In each case, the fracture requires stability and compression of bone fragments together such that the patient's patella 16 may properly heal.

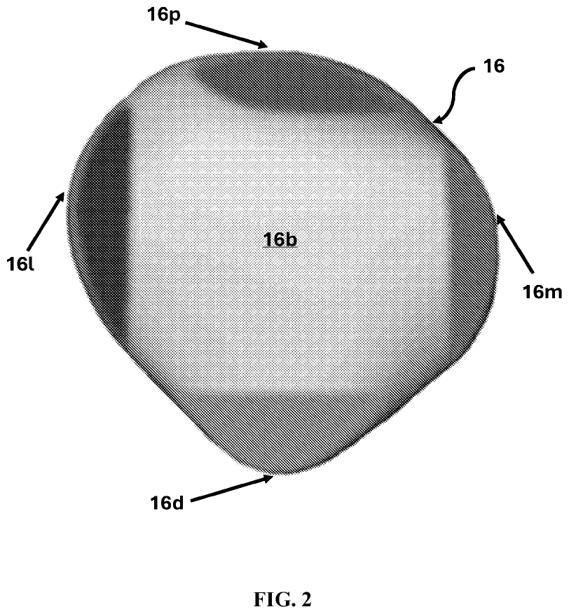

FIG. 2 illustrates the anterior surface 16b of a typical patella 16. The anterior surface 16b may include a proximal portion 16p (e.g., a portion of the patella 16 in proximity to the quadriceps tendon 18). The anterior surface 16b may further include a distal portion 16d (e.g., a portion of the patella 16 in proximity to the patella tendon 20) opposite of the proximal portion 16p. The anterior surface 16b may further include a lateral side portion 16l (e.g., a portion of the patella 16 in proximity to its lateral border) and a medial side portion 16m (e.g., a portion of the patella 16 in proximity to its medial border) opposite the lateral side portion 16l.

Embodiments relate to a patella reinforcing member 100 configured to stabilize a fracture and deliver compression between bone fragments. As will be described in further detail, the member 100 is configured as a compliant mechanism, which can be defined as a mechanism that achieves movement and force transmission through elastic deformation (e.g., stretching) of flexible components. For example, the member 100 may be pre-tensioned/stretched to fit around the patella 16. The member 100 may then stabilize and compress bone fragments as it releases the pre-tensioning force to return to its initial, non-tensioned state. Due to its flexible nature, the patella reinforcing member 100 is also configured to accommodate high tensile loading of the patella 16.

As used herein, the term “patient” may refer to any biological system in which the patella reinforcing member 100 can be implanted, including without limitation, humans and other animals (e.g., dogs, cats, horses, cows, cattle, etc.).

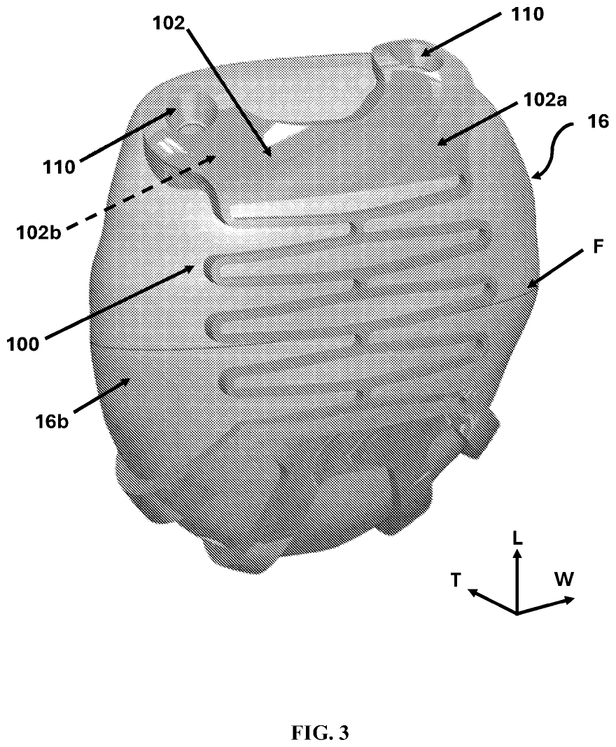

Referring to FIG. 3, the patella reinforcing member 100 includes a body 102 having a first surface 102a and an opposite second surface 102b, wherein the first and second surfaces 102a, 102b are separated by a thickness of the body 102. The second surface 102b is an inward facing surface configured to abut the anterior surface 16b of the patella 16. The second surface 102b may have any shape and contour, though it is preferably shaped to complement the anterior surface 16b of the patella 16, which is typically convex/rounded. The second surface 102b may therefore similarly be convex/rounded. The first surface 102a is an outward facing surface and may have any shape and contour. For example, the first surface 102a may be planar (e.g., flat), concave, convex, or a combination thereof. In some embodiments, the first surface 102a may be shaped to match or complement the shape of the second surface 102b. In some embodiments, the first surface 102a may be shaped to match or complement the anterior surface 16b of the patella 16, such that the first surface 102a may allow the body 102 to have a naturally shaped outward facing surface.

The body 102 has a length extending in a longitudinal direction L, a thickness extending in a thickness direction T, and a width extending in a transverse direction W. The transverse direction is perpendicular to both the longitudinal and thickness directions.

The body 102 may be made from a rigid material, such as a metal (e.g., titanium, magnesium), metal alloy (e.g., stainless steel, titanium alloy, magnesium alloy), polymeric material, composites, and/or combinations thereof. It is contemplated that while the material of the body 102 may be rigid, the body may form a flexible geometry to accommodate the compliant nature of the patella reinforcing member 100. In some embodiments, the body 102 is made from a biocompatible material. In some embodiments, the body 102 is bioresorbable in such that biodegradation kinetics may be tuned to facilitate degradation of the member 100 over time.

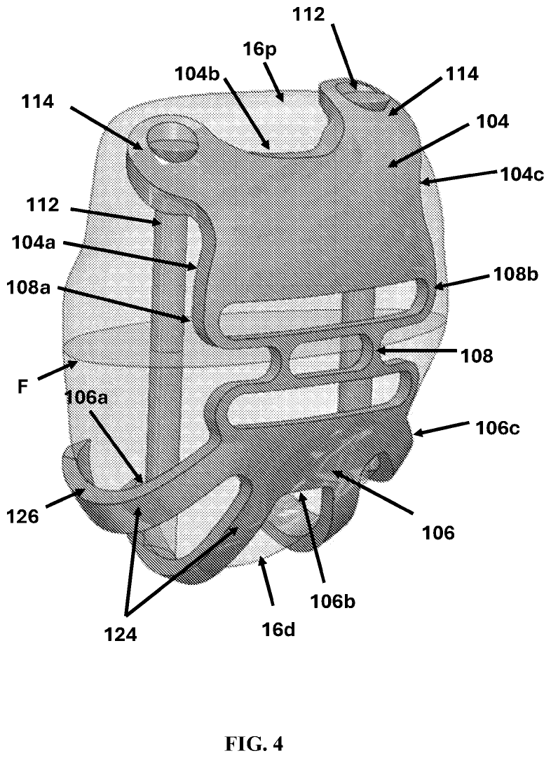

Referring to FIGS. 3-4, a first exemplary embodiment of the body 102 configured to deliver primarily single-plane compression to the patella 16 may include a first portion 104 and a longitudinally opposite second portion 106. The term “single-plane compression” means that the body 102 may deliver compression primarily linearly, such as either in a horizontal direction, a vertical direction, etc.

The first portion 104 may be configured to anchor to and/or engage a proximal portion 16p of the patella 16. The second portion 106 may be configured to anchor to and/or engage a distal portion 16d of the patella 16.

The first portion 104 may be defined by a plurality of edges. For example, the first portion 104 may include one or more first side edges 104a, one or more second side edges 104c transversely opposite the one or more first side edges 104a, and one or more end edges 104b positioned between and connected the first 104a and second 104c side edges. The edges may be any type of edge, such as straight, curved, sharp, rounded, and combinations thereof.

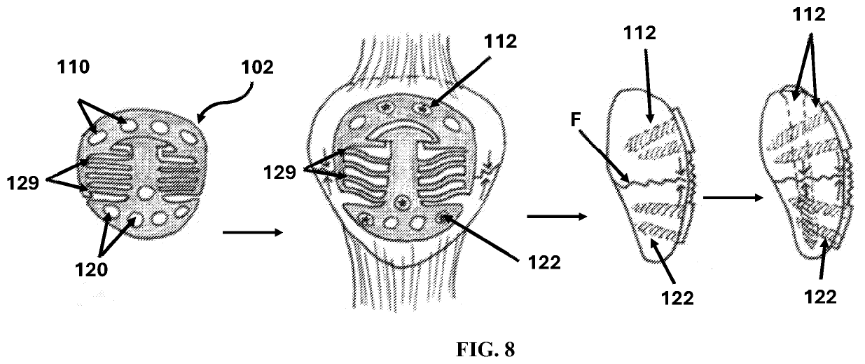

The first portion 104 may include a plurality of holes 110 extending from the first surface 102a through the second surface 102b. The holes 110 can be configured to receive fasteners 112 to anchor the first portion 104 to the patella 16, and/or to receive wire, suture, cerclage wire, and combinations thereof (not shown) configured to compress the body 102 (e.g., compress the first portion 104 towards the second portion 106). In some embodiments, the holes 110 may be spaced apart and arranged in a plurality of positions within the first portion 104 (see e.g., FIGS. 3 and 8). By providing spaced apart holes 110 in a plurality of positions, the body 102 may provide for flexibility in positioning the member 100 in relation to the patella 16. Further, providing spaced apart holes 110 in a plurality of positions may allow for anchoring of the first portion 104 to the patella via fasteners 112 at different positions, which may help distribute loads and/or secure the entirety of the first portion 104 to the patella 16. Even further, providing spaced apart holes 110 in a plurality of positions may provide for flexibility in how a wire, suture, cerclage wire, and combinations thereof may be thread throughout the body 102.

The first portion 104 may include any number of holes 110. In some embodiments, the first portion 104 includes one hole, two holes, three holes, four holes, five holes, six holes, seven holes, eight holes, nine holes, ten holes, etc.

In some embodiments, the first portion 104 may include at least one hole 110 positioned near the one or more first side edges 104a and at least one hole 110 positioned near the one or more second side edges 104c. In some embodiments, the first portion 104 may include at least one hole 110 positioned near the one or more first side edges 104a, at least one hole 110 positioned between, such as centrally located between, the first and second side edges 104a, 104c, and at least one hole 110 positioned near the one or more second side edges 104c.

The first portion 104 may include one or more projections 114 extending from the one or more end edges 104b in the longitudinal L and/or thickness T directions. For example, the projections 114 may be convex/rounded so as to conform with the shape of the proximal portion 16p of the patella 16. The projections 114 may extend onto or around the proximal portion 16p (e.g., onto or around the superior surface of the patella).

The first portion 104 may include any number of projections 114. In some embodiments, the first portion 104 includes one projection, two projections, three projections, four projections, five projections, six projections, etc.

In some embodiments, the first portion 104 may include at least one projection 114 positioned at or near the one or more first side edges 104a and at least one projection 114 positioned at or near the one or more second side edges 104c. In some embodiments, the first portion 104 may include at least one projection 114 positioned at or near the one or more first side edges 104a, at least one projection 114 positioned between, such as centrally located between, the first and second side edges 104a, 104c, and at least one projection 114 positioned at or near the one or more second side edges 104c. In some embodiments, the first portion 104 may include at least one projection 114 positioned at or near the one or more first side edges 104a, two or more projections 114 positioned between the first and second side edges 104a, 104c, and at least one projection 114 positioned at or near the one or more second side edges 104c.

In some embodiments, at least one of the projections 114 may include a hole 110 configured to receive a fastener 112 to anchor the first portion 104 to the patella 16. In some embodiments, some or all of the projections 114 may include a hole 110 configured to receive a fastener 112 to anchor the first portion 104 to the patella 16.

In some embodiments, the first portion 104 may include at least one projection 114 with a hole 110 positioned at or near the one or more first side edges 104a and at least one projection 114 with a hole 110 positioned at or near the one or more second side edges 104c.

The fasteners 112 used in the first portion 104 may be screws, bolts, or any other suitable fastener and/or combination thereof.

The fasteners 112 may be configured to as lateral fasteners (e.g., lateral screws) such that the fasteners 112 extend into the anterior surface 16b of the patella 16 in or substantially in a perpendicular manner.

The fasteners 112 may alternatively or additionally be configured as lag fasteners (e.g., lag screws) such that the fasteners 112 extend into the anterior surface 16b of the patella 16 at an angle. Lag fasteners are typically configured to provide compression between two fragments of bone, and is therefore contemplated that the lag fasteners may extend into and/or through more than one bone fragment.

In some embodiments, at least one of the projections 114 may be configured as a hook (not shown). The hook may be configured to fit around the patella 16, such that the first portion 104 grasps onto the proximal portion 16p of the patella 16 to anchor the first portion 104 to the patella 16. For example, the hook may extend onto and around the superior surface of the patella 16.

In some embodiments, the first portion 104 may include at least one hook positioned at or near the one or more first side edges 104a and at least one hook positioned at or near the one or more second side edges 104c. In some embodiments, the first portion 104 may include at least one hook positioned at or near the one or more first side edges 104a, at least one hook positioned between, such as centrally located between, the first and second side edges 104a, 104c, and at least one hook positioned at or near the one or more second side edges 104c. In some embodiments, the first portion 104 may include at least one hook positioned at or near the one or more first side edges 104a, two or more hooks positioned between the first and second side edges 104a, 104c, and at least one hook positioned at or near the one or more second side edges 104c.

The first portion 104 may therefore be anchored to the patella 16 via fasteners 112, hooks, or a combination thereof. In preferred embodiments, the first portion 104 may be anchored to the patella without removing bone mass or disrupting soft tissue.

The second portion 106 may be defined by a plurality of edges. For example, the second portion 106 may include one or more first side edges 106a, one or more second side edges 106c transversely opposite the one or more first side edges 106a, and one or more end edges 106b positioned between and connected the first 106a and second 106c side edges. The edges may be any type of edge, such as straight, curved, sharp, rounded, and combinations thereof.

The second portion 106 may include a plurality of holes 120 extending from the first surface 102a through the second surface 102b (see e.g., FIG. 8). The holes 120 can be configured to receive fasteners 122 to anchor the second portion 106 to the patella 16, and/or to receive wire, suture, cerclage wire, and combinations thereof (not shown) configured to compress the body 102 (e.g., compress the second portion 106 towards the first portion 104). In some embodiments, the holes 120 may be spaced apart and arranged in a plurality of positions within the second portion 106. By providing spaced apart holes 120 in a plurality of positions, the body 102 may provide for flexibility in positioning the member 100 in relation to the patella 16. Further, providing spaced apart holes 120 in a plurality of positions may allow for anchoring the second portion 106 to the patella via fasteners 122 at different positions, which may help distribute loads and/or secure the entirety of the second portion 106 to the patella 16. Even further, providing spaced apart holes 120 in a plurality of positions may provide for flexibility in how a wire, suture, cerclage wire, and combinations thereof may be thread throughout the body 102.

The second portion 106 may include any number of holes 120. In some embodiments, the second portion 106 includes one hole, two holes, three holes, four holes, five holes, six holes, seven holes, eight holes, nine holes, ten holes, etc.

In some embodiments, the second portion 106 may include at least one hole 120 positioned near the one or more first side edges 106a and at least one hole 120 positioned near the one or more second side edges 106c. In some embodiments, the second portion 106 may include at least one hole 120 positioned near the one or more first side edges 106a, at least one hole 120 positioned between, such as centrally located between, the first and second side edges 106a, 106c, and at least one hole 120 positioned near the one or more second side edges 106c.

The second portion 106 may include one or more projections 124 extending from the one or more end edges 106b in the longitudinal and/or thickness directions. For example, the projections 124 may be convex/rounded so as to conform with the shape of the distal portion 16d of the patella 16. The projections 124 may extend onto or around the distal portion 16d (e.g., onto or around the bottom apex of the patella).

The second portion 106 may include any number of projections 124. In some embodiments, the second portion 106 includes one projection, two projections, three projections, four projections, five projections, six projections, etc.

In some embodiments, the second portion 106 may include at least one projection 124 positioned at or near the one or more first side edges 106a and at least one projection 124 positioned at or near the one or more second side edges 106c. In some embodiments, the second portion 106 may include at least one projection 124 positioned at or near the one or more first side edges 106a, at least one projection 124 positioned between, such as centrally located between, the first and second side edges 106a, 106c, and at least one projection 124 positioned at or near the one or more second side edges 106c. In some embodiments, the second portion 106 may include at least one projection 124 positioned at or near the one or more first side edges 106a, two or more projections 124 positioned between the first and second side edges 106a, 106c, and at least one projection 124 positioned at or near the one or more second side edges 106c.

In some embodiments, at least one of the projections 124 includes a hole 120 configured to receive a fastener 122 to anchor the second portion 106 to the patella 16. In some embodiments, some or all of the projections 124 include a hole 120 configured to receive a fastener 122 to anchor the second portion 106 to the patella 16.

In some embodiments, the second portion 106 may include at least one projection 124 with a hole 120 positioned at or near the one or more first side edges 106a and at least one projection 124 with a hole 120 positioned at or near the one or more second side edges 106c.

The fasteners 122 used in the second portion 106 may be screws, bolts, or any other suitable fastener and/or combination thereof. Fasteners 122 used in the first portion 104 and fasteners 122 used in the second portion 106 may be the same or different.

The fasteners 122 may be configured to as lateral fasteners (e.g., lateral screws) such that the fasteners 122 extend into the anterior surface 16b of the patella 16 in or substantially in a perpendicular manner.

The fasteners 122 may alternatively or additionally be configured as lag fasteners (e.g., lag screws) such that the fasteners 122 extend into the anterior surface 16b of the patella 16 at an angle. Lag fasteners are typically configured to provide compression between two fragments of bone, and is therefore contemplated that the lag fasteners may extend into and/or through more than one bone fragment.

In some embodiments, at least one of the projections 124 may be configured as a hook 126. The hook 126 may be configured to fit around the patella 16, such that the second portion 106 grasps onto or around the distal portion 16d of the patella 16 to anchor the second portion 106 to the patella 16.

In some embodiments, the second portion 106 may include at least one hook 126 positioned near or at the one or more first side edges 106a and at least one hook 126 positioned near or at the one or more second side edges 106c. In some embodiments, the second portion 106 may include at least one hook 126 positioned near or at the one or more first side edges 106a, at least one hook 126 positioned between, such as centrally located between, the first and second side edges 106a, 106c, and at least one hook 126 positioned near or at the one or more second side edges 106c. In some embodiments, the second portion 106 may include at least one hook 126 positioned near or at the one or more first side edges 106a, two or more hooks 126 positioned between the first and second side edges 106a, 106c, and at least one hook 126 positioned near or at the one or more second side edges 106c.

The second portion 106 may therefore be anchored to the patella 116 via fasteners 122, hooks 126, or a combination thereof. In preferred embodiments, the second portion 106 may be anchored to the patella without removing bone mass or disrupting soft tissue.

The body 102 further includes an intermediate portion 108 positioned between the first portion 104 and the second portion 106. The intermediate portion 108 may be configured to engage a central portion of the anterior surface 16b of the patella 16.

The intermediate portion 108 is stretchable such that it may be pre-tensioned and movable from an initial position to one or more stretched positions. The initial position refers to an equilibrium (e.g., non-tensioned) state in which the intermediate portion 108 is neither stretched nor flexed and experiences a net zero force. The one or more stretched positions refer to a position after application of various levels of stretching to the intermediate portion 108. The intermediate portion 108 may therefore be movable from its initial position to one or more stretched positions by pre-tensioning/stretching the intermediate portion 108. In particular, the intermediate portion 108 is configured to be resiliently movable from its initial position to one or more stretched positions, such that the intermediate portion 108 may return to its initial position after being stretched, thereby compressing the first portion 104 and the second portion 106 towards one another. The intermediate portion 108 may further be configured to be resiliently biased to return to its initial position after being stretched.

The intermediate portion 108 may therefore enable the member 100 to be configured as a compliant mechanism, which can be defined as a mechanism that achieves movement and force transmission through elastic deformation (e.g., stretching) of flexible components, such as the intermediate portion 108. Due the stretchable nature of the intermediate portion 108, the member 100 may be pre-tensioned/stretched to fit around the patella 16, and the member 100 may then compress bone fragments (e.g., by linearly compressing the first portion 104 and the second portion 106 towards one another) as it releases the pre-tensioning force within the intermediate portion 108 to return to its initial position.

The intermediate portion 108 may be defined by a flexible topology positioned between the first portion 104 and the second portion 106. The flexible topology may include one or more first side edges 108a connecting the first portion 104 to the second portion 106, and one or more second side edges 108b transversely opposite the one or more first side edges 108a connecting the first portion 104 to the second portion 106.

The flexible topology enables the intermediate portion 108 to be pre-tensioned/stretched from an initial position to one or more stretched positions as described above. The flexible topology may specifically be configured for linear motion such that the first portion 104 and the second portion 106 are pulled towards one another. Such linear motion may be particularly beneficial for transversal and/or comminuted patella fractures.

In some embodiments, the flexible topology may include a compliant array resembling a lamina emergent torsional joint (shown in FIGS. 3-6).

The flexible topology may include an array of openings 128 (e.g., honeycomb pattern, re-entrant honeycomb pattern, polygonal shapes, auxetic shapes, etc.), serpentine flexures, straight flexures, zigzag flexures, lattice structures, topology-optimized structure, or any flexible geometry that facilitates and/or promotes elastic deflection.

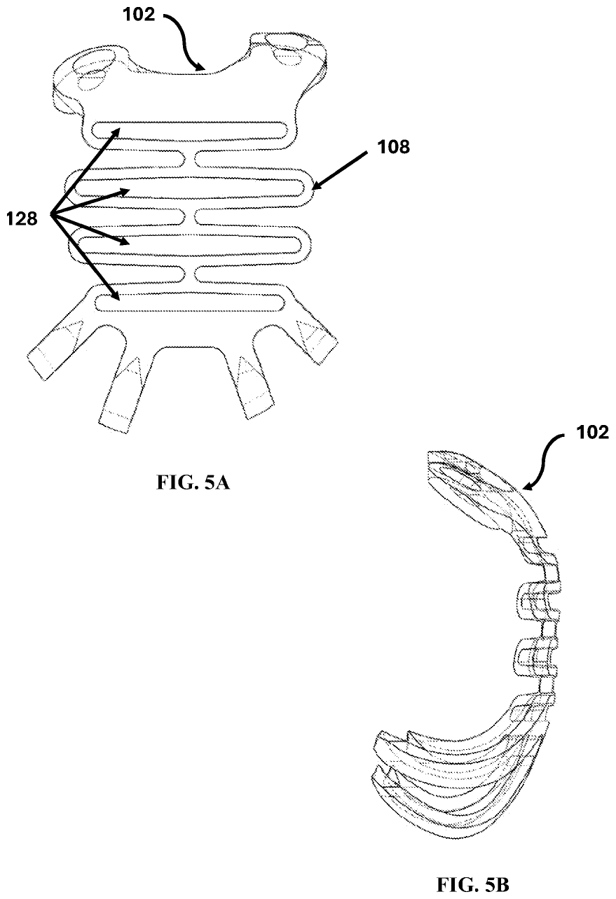

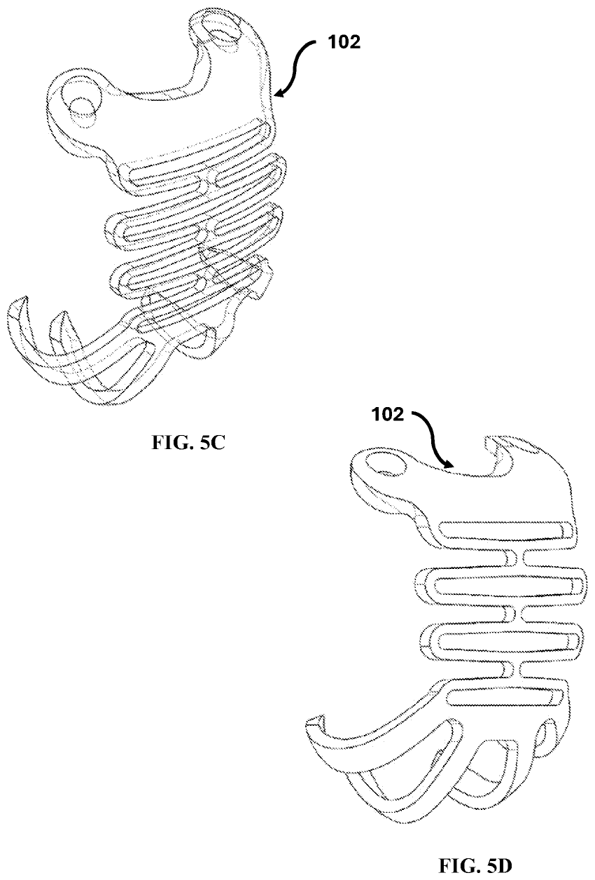

Each opening 128 may have a first initial size when the intermediate portion is in its initial position and a second, larger size when the intermediate portion is in one of its stretched positions. Referring to FIGS. 5A-7, the array of openings 128 can include any number of openings 128. In some embodiments, the intermediate portion 108 may include one opening, two openings, three openings, four openings, five openings, six openings, seven openings, eight openings, nine openings, ten openings, etc., though the number of openings 128 are not particularly limited as long as the openings 128 enable a flexible geometry. In some embodiments, the openings may have a uniform initial size and/or shape. In alternative embodiments, the openings 128 may not have a uniform initial size and/or shape. For example, the openings 128 may include any number of unique initial sizes/shapes. In some embodiments, at least one opening has a first initial size, at least one opening has a second initial size, at least one opening has a third initial size, at least one opening has a fourth initial size, etc.

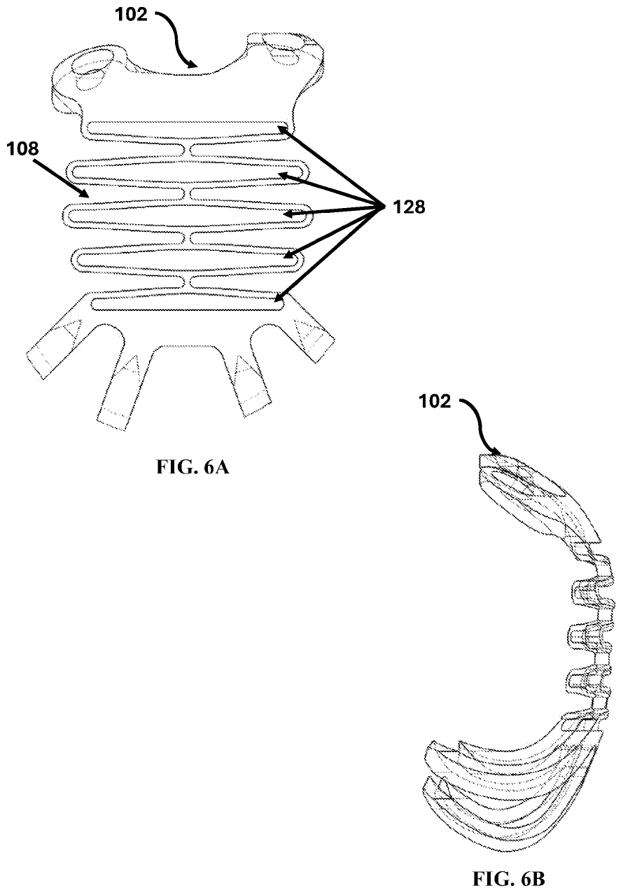

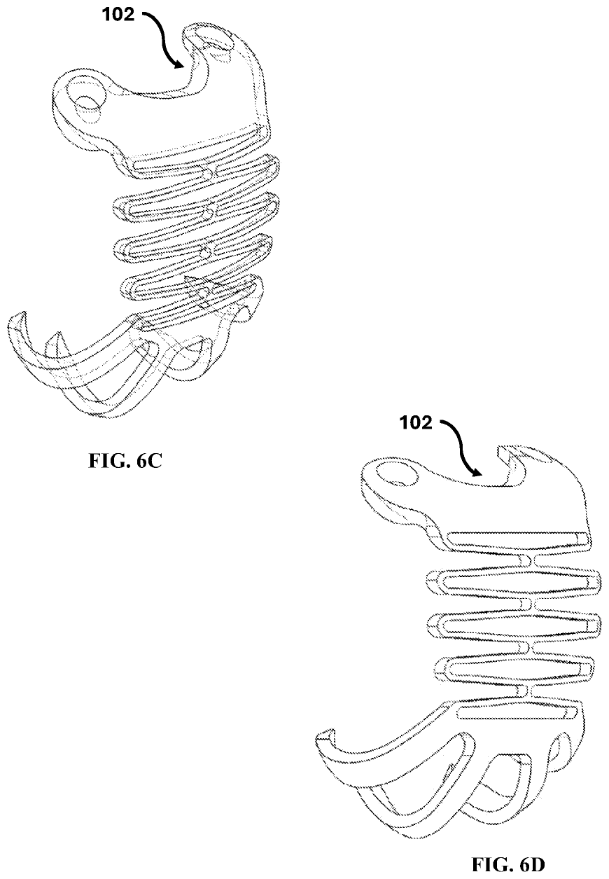

The array of openings 128 may be positioned in any configuration so long as the openings enable a flexible geometry. Referring to FIGS. 5A-6D, the openings 128 may be positioned in a plurality of rows, wherein each row has a singular opening. For example, in some embodiments, a first segment defining a first opening adjacent the first portion 104 may have a first initial size when the intermediate portion 108 is in the initial position and may have a second larger size when the intermediate portion 108 is in a stretched position. Moreover, a second segment defining a second opening adjacent the second portion 106 may have a first initial size when the intermediate portion 108 is in the initial position and may have a second larger size when the intermediate portion 108 is in a stretched position. There may further be third, fourth, fifth, sixth, seventh, eighth, ninth, tenth, etc. segment positioned between the first and second segments with first initial sizes and second larger sizes.

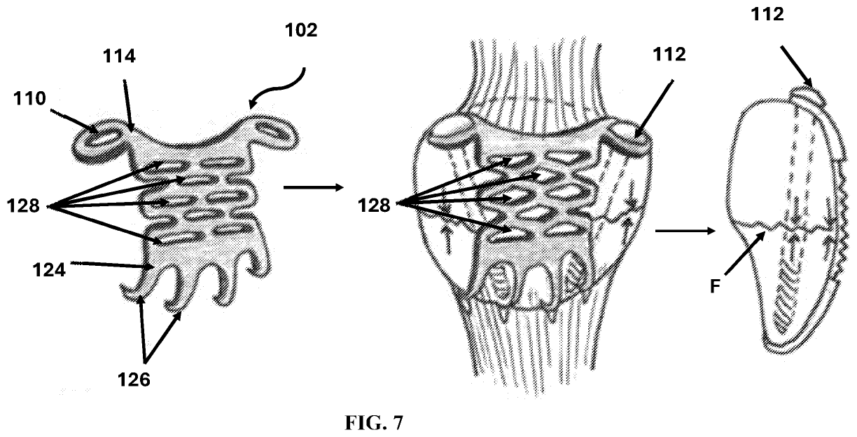

Alternatively, referring to FIG. 7, the openings 128 may be positioned in a plurality of rows, wherein at least one row has a plurality of openings.

In some embodiments, the array of openings 128 may be configured in a honeycomb pattern. The honeycomb pattern may include a plurality of adjacently arranged openings cells. The honeycomb pattern can include any number of cells, and the number of cells is not particularly limited as long as the honeycomb pattern enables a flexible geometry. In some embodiments, the cells may be uniform hexagonal (six-sided) cells such that the cells may be arranged together without any gaps.

In some embodiments, the array of openings 128 may be configured as a re-entrant honeycomb pattern. The re-entrant honeycomb pattern may include a plurality of adjacently arranged openings/cells (as in the honeycomb pattern), though may further include a plurality of inward curves or indentations (e.g., rather than straight) edges in its cells. The re-entrant honeycomb pattern can include any number of cells, and the number of cells is not particularly limited as long as the re-entrant honeycomb pattern enables a flexible geometry. The cells may nevertheless be uniform hexagonal (six-sided) cells such that the cells may be arranged together without any gaps.

In some embodiments, the array of openings 128 may be configured as a pattern of polygonal shapes. The polygonal pattern may include a plurality of adjacently arranged polygonal openings/cells. In some embodiments, the polygonal cells may include without limitation triangular (three-sided), quadrilateral (four-sided), pentagonal (five-sided), hexagonal (six-sided), heptagonal (seven-sided), octagonal (eight-sided), nonagonal (nine-sided), decagonal (ten-sided) cells, etc., or combinations thereof. The polygonal pattern can include any number of cells, and the number of cells is not particularly limited as long as the polygonal pattern enables a flexible geometry. The polygonal cells may be uniform and arranged together without any gaps.

In some embodiments, the array of openings 128 may be configured as a pattern of auxetic shapes. The auxetic pattern may include a plurality of adjacently arranged auxetic openings/cells. The term “auxetic” refers to the cells ability to expand (e.g., rather than contract) in one direction as the cells are stretched in another direction. The auxetic pattern can include any number of cells, and the number of cells is not particularly limited as long as the auxetic pattern enables a flexible geometry. The auxetic cells may be uniform and arranged together without any gaps.

In some embodiments, the flexible topology may include flexures 129 (see FIG. 8), such as straight flexures. The term “straight flexures” refers to initially straight components configured to elastically bend under load, and the term “linear motion reinforcement elements” refers to components configured to allow flexure deflection in a desired linear direction, while preventing deflection in undesired directions, such as torsional motion. Put differently, when a force is applied to a straight flexure, the flexure may bend and allows for controlled movement, and the reinforcement element may help maintain the shape and integrity of the flexure, while also maintaining a primarily linear direction of motion. Each straight flexure may have a first initial size/shape when the intermediate portion is in its initial position and a second, larger size/shape when the intermediate portion is in one of its stretched positions. The flexible topology may include any number of straight flexures, such as one straight flexure, two straight flexures, three straight flexures, four straight flexures, five straight flexures, six straight flexures, seven straight flexures, eight straight flexures, nine straight flexures, ten straight flexures, etc., though the number of straight flexures is not particularly limited as long as the flexures enable a flexible geometry.

In some embodiments, overall expansion of the member 100 may be facilitated through compression of flexures (e.g., rather than just flexure expansion/stretching).

In some embodiments, the flexible topology may include serpentine flexures. The term “serpentine flexures” refers to a series of curved bends or loops that are designed to bend and stretch efficiently. In some embodiments, the flexible geometry may include one or more fused serpentine flexures in parallel. Each serpentine flexure may have a first initial size/shape when the intermediate portion is in its initial position and a second, larger size/shape when the intermediate portion is in one of its stretched positions. The flexible topology may include any number of serpentine flexures, such as one serpentine flexure, two serpentine flexures, three serpentine flexures, four serpentine flexures, five serpentine flexures, six serpentine flexures, seven serpentine flexures, eight serpentine flexures, nine serpentine flexures, ten serpentine flexures, etc., though the number of serpentine flexures is not particularly limited as long as the flexures enable a flexible geometry.

In some embodiments, the flexible topology may include zigzag flexures with torsional reinforcement elements. The term “zigzag flexures” refers to a series of sharp bends or angles, thus resembling a zigzag pattern, and may be configured to flex or bend along the zigzag shape when a force is applied. Each zigzag flexure may have a first initial size/shape when the intermediate portion is in its initial position and a second, larger size/shape when the intermediate portion is in one of its stretched positions. The flexible topology may include any number of zigzag flexures, such as one zigzag flexure, two zigzag flexures, three zigzag flexures, four zigzag flexures, five zigzag flexures, six zigzag flexures, seven zigzag flexures, eight zigzag flexures, nine zigzag flexures, ten zigzag flexures, etc., though the number of zigzag flexures is not particularly limited as long as the flexures enable a flexible geometry.

In some embodiments, the flexible topology may include lattice structures. A lattice structure may include a plurality of adjacently arranged, interconnected cells that form a grid-like pattern. In particular, the lattice structure may include a plurality of straight lines that connect at various points to create a network, and the connections often form geometric shapes (e.g., triangles, quadrilaterals, etc.), thus forming a grid-like pattern.

In some embodiments, the flexible topology may include any topology-optimized geometries that facilitates and/or promotes linear compression between the first 104 and second 106 portions.

In some embodiments, the flexible topology includes a combination of two or more topologies described above.

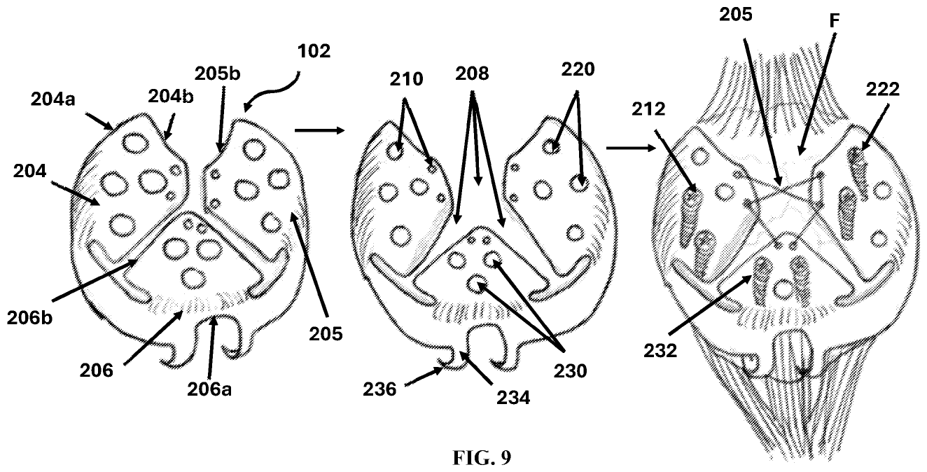

Referring to FIG. 9, a second exemplary embodiment of the body 102 configured to deliver multi-plane compression to the patella 16 may include a first side portion 204, a transversely opposite second side portion 205, and a third portion 206 positioned between and connecting the first side portion 204 and the second side portion 205. The term “multi-plane compression” means that the body 102 may deliver compression in more than one direction, such as in a horizontal direction and a vertical direction, an inward radial direction, etc.

The first side portion 204 may be configured to anchor to and/or engage a lateral side portion 16l of the patella 16. The second side portion 205 may be configured to anchor to and/or engage a medial side portion 16m of the patella 16. The third portion 206 may be configured to anchor to and/or engage a distal portion 16d of the patella 16.

The first side portion 204 may be defined by one or more edges. For example, the first side portion 204 may include one or more outer edges 204a and one or more inner edges 204b. The edges may be any type of edge, such as straight, curved, sharp, rounded, and combinations thereof.

The second side portion 205 may be defined by one or more edges. For example, the second side portion 205 may include one or more outer edges 205a and one or more inner edges 205b. The edges may be any type of edge, such as straight, curved, sharp, rounded, and combinations thereof.

The third portion 206 may be defined by one or more edges. For example, the third portion 206 may include one or more outer edges 206a and one or more inner edges 206b. The outer edges 206a may be positioned between and connected to the outer edges of the first 204a and second 205a side portions, and the inner edges 206b may be connected to the inner edges of the first 204b and second 205b side portions. The edges may be any type of edge, such as straight, curved, sharp, rounded, and combinations thereof.

The first side portion 204 may include a plurality of holes 210 extending through the body 102. The holes 210 can be configured to receive fasteners 212 to anchor the first side portion 204 to the patella 16, and/or to receive wire 205 (or suture, cerclage wire, and combinations thereof) configured to compress the body 102. In some embodiments, the holes 210 may be spaced apart and arranged in a plurality of positions within the first side portion 204. By providing spaced apart holes 210 in a plurality of positions, the body 102 may provide for flexibility in positioning the member 100 in relation to the patella 16. Further, providing spaced apart holes 210 in a plurality of positions may allow for anchoring of the first side portion 204 to the patella via fasteners 212 at different positions, which may help distribute loads and/or secure the entirety of the first side portion 204 to the patella 16. Even further, providing spaced apart holes 210 in a plurality of positions may provide for flexibility in how a wire 205 (or suture, cerclage wire, and combinations thereof) may be thread throughout the body 102.

The first side portion 204 may include any number of holes 210. In some embodiments, the first side portion 204 includes one hole, two holes, three holes, four holes, five holes, six holes, seven holes, eight holes, nine holes, ten holes, etc.

The first side portion 204 may include one or more projections (not shown) extending from the one or more outer edges 204a in the transverse and/or thickness directions. For example, the projections may be convex/rounded so as to conform with the shape of the lateral side portion 161 of the patella 16.

The first side portion 204 may include any number of projections. In some embodiments, the first side portion 204 includes one projection, two projections, three projections, four projections, five projections, six projections, etc.

In some embodiments, at least one of the projections may include a hole 210 configured to receive a fastener 212 to anchor the first side portion 204 to the patella 16. In some embodiments, some or all of the projections may include a hole 210 configured to receive a fastener 212 to anchor the first side portion 204 to the patella 16.

The fasteners 212 used in the first side portion 204 may be screws, bolts, or any other suitable fastener and/or combination thereof.

The fasteners 212 may be configured to as lateral fasteners (e.g., lateral screws) such that the fasteners 212 extend into the anterior surface 16b of the patella 16 in or substantially in a perpendicular manner.

The fasteners 212 may alternatively or additionally be configured as lag fasteners (e.g., lag screws) such that the fasteners 212 extend into the anterior surface 16b of the patella 16 at an angle. Lag fasteners are typically configured to provide compression between two fragments of bone, and is therefore contemplated that the lag fasteners may extend into and/or through more than one bone fragment.

In some embodiments, at least one of the projections may be configured as a hook (not shown). The hook may be configured to fit around the patella 16, such that the first side portion 204 grasps a lateral side portion 16l the patella 16 to anchor the first side portion 204 to the patella 16.

The first side portion 204 may therefore be anchored to the patella 16 via fasteners 212, hooks 216, or a combination thereof. In preferred embodiments, the first side portion 204 may be anchored to the patella without removing bone mass or disrupting soft tissue.

The second side portion 205 may include a plurality of holes 220 extending through the body 102. The holes 220 can be configured to receive fasteners 222 to anchor the second side portion 205 to the patella 16, and/or to receive wire 205 (or suture, cerclage wire, and combinations thereof) configured to compress the body 102. In some embodiments, the holes 220 may be spaced apart and arranged in a plurality of positions within the second side portion 205. By providing spaced apart holes 220 in a plurality of positions, the body 102 may provide for flexibility in positioning the member 100 in relation to the patella 16. Further, providing spaced apart holes 220 in a plurality of positions may allow for anchoring of the second side portion 205 to the patella via fasteners 222 at different positions, which may help distribute loads and/or secure the entirety of the second side portion 205 to the patella 16. Even further, providing spaced apart holes 220 in a plurality of positions may provide for flexibility in how a wire 205 (or suture, cerclage wire, and combinations thereof) may be thread throughout the body 102.

The second side portion 205 may include any number of holes 220. In some embodiments, the second side portion 205 includes one hole, two holes, three holes, four holes, five holes, six holes, seven holes, eight holes, nine holes, ten holes, etc.

The second side portion 205 may include one or more projections (not shown) extending from the one or more outer edges 205a in the transverse and/or thickness directions. For example, the projections may be convex/rounded so as to conform with the shape of the medial side portion 16m of the patella 16.

The second side portion 205 may include any number of projections. In some embodiments, the second side portion 205 includes one projection, two projections, three projections, four projections, five projections, six projections, etc.

In some embodiments, at least one of the projections may include a hole 220 configured to receive a fastener 222 to anchor the second side portion 205 to the patella 16. In some embodiments, some or all of the projections may include a hole 220 configured to receive a fastener 222 to anchor the second side portion 205 to the patella 16.

The fasteners 222 used in the second side portion 205 may be screws, bolts, or any other suitable fastener and/or combination thereof.

The fasteners 222 may be configured to as lateral fasteners (e.g., lateral screws) such that the fasteners 222 extend into the anterior surface 16b of the patella 16 in or substantially in a perpendicular manner.

The fasteners 222 may alternatively or additionally be configured as lag fasteners (e.g., lag screws) such that the fasteners 222 extend into the anterior surface 16b of the patella 16 at an angle. Lag fasteners are typically configured to provide compression between two fragments of bone, and is therefore contemplated that the lag fasteners may extend into and/or through more than one bone fragment.

In some embodiments, at least one of the projections may be configured as a hook (not shown). The hook may be configured to fit around the patella 16, such that the second side portion 205 grasps a medial side portion 16m of the patella 16 to anchor the second side portion 205 to the patella 16. For example, the hook may extend onto and around the medial border of the patella 16.

The third portion 206 may include a plurality of holes 230 extending through the body 102. The holes 230 can be configured to receive fasteners 232 to anchor the third portion 206 to the patella 16, and/or to receive wire 205 (or suture, cerclage wire, and combinations thereof) configured to compress the body 102. In some embodiments, the holes 230 may be spaced apart and arranged in a plurality of positions within the third portion 206. By providing spaced apart holes 230 in a plurality of positions, the body 102 may provide for flexibility in positioning the member 100 in relation to the patella 16. Further, providing spaced apart holes 230 in a plurality of positions may allow for anchoring of the third portion 206 to the patella via fasteners 232 at different positions, which may help distribute loads and/or secure the entirety of the third portion 206 to the patella 16. Even further, providing spaced apart holes 230 in a plurality of positions may provide for flexibility in how a wire 205 (or suture, cerclage wire, and combinations thereof) may be thread throughout the body 102.

The third portion 206 may include any number of holes 230. In some embodiments, the third portion 206 includes one hole, two holes, three holes, four holes, five holes, six holes, seven holes, eight holes, nine holes, ten holes, etc.

The third portion 206 may include one or more projections 234 extending from the one or more outer edges 206a in the longitudinal and/or thickness directions. For example, the projections 234 may be convex/rounded so as to conform with the shape of the distal portion 16d of the patella 16.

The third portion 206 may include any number of projections 234. In some embodiments, the third portion 206 includes one projection, two projections, three projections, four projections, five projections, six projections, etc.

In some embodiments, at least one of the projections 234 may include a hole 230 configured to receive a fastener 232 to anchor the third portion 206 to the patella 16. In some embodiments, some or all of the projections 234 may include a hole 230 configured to receive a fastener 232 to anchor the third portion 206 to the patella 16.

The fasteners 232 used in the third portion 206 may be screws, bolts, or any other suitable fastener and/or combination thereof.

The fasteners 232 may be configured to as lateral fasteners (e.g., lateral screws) such that the fasteners 232 extend into the anterior surface 16b of the patella 16 in or substantially in a perpendicular manner.

The fasteners 232 may alternatively or additionally be configured as lag fasteners (e.g., lag screws) such that the fasteners 232 extend into the anterior surface 16b of the patella 16 at an angle. Lag fasteners are typically configured to provide compression between two fragments of bone, and is therefore contemplated that the lag fasteners may extend into and/or through more than one bone fragment.

In some embodiments, at least one of the projections 234 may be configured as a hook 236. The hook 236 may be configured to fit around the patella 16, such that the third portion 206 grasps the distal portion 16d of the patella 16 to anchor the third portion 206 to the patella 16.

The third portion 206 may therefore be anchored to the patella 16 via fasteners 232, hooks 236, or a combination thereof. In preferred embodiments, the third portion 206 may be anchored to the patella without removing bone mass or disrupting soft tissue.

The body 102 further includes an intermediate portion including one or more channels 208. The channels may be positioned between the first side portion 204, the second side portion 205, and/or the third portion 206 such that their inner portions are separated.

The one or more channels 208 are configured to enable the body 102 to be stretchable such that it may be pre-tensioned and movable from an initial position to one or more stretched positions. The initial position refers to an equilibrium (e.g., non-tensioned) state in which the first side portion 204, the second side portion 205, and the third portion 206 are neither stretched nor flexed and experiences a net zero force. The one or more stretched positions refer to a position after application of various levels of stretching to the body 102. The body 102 may therefore be movable from its initial position to one or more stretched positions by pre-tensioning/stretching at least one of the first side portion 204, the second side portion 205, and/or the third portion 206. In particular, at least one of the first side portion 204, the second side portion 205, and/or the third portion 206 is configured to be resiliently movable from an initial position to one or more stretched positions, such that the portion may return to its initial position after being stretched, thereby compressing the body 102 inward. The body 102 may further be configured to be resiliently biased to return to its initial position after being stretched.

The member 100 may therefore be configured as a compliant mechanism. Due the stretchable nature of at least one of the first side portion 204, the second side portion 205, and/or the third portion 206, the member 100 may be pre-tensioned/stretched to fit around the patella 16, and the member 100 may then compress bone fragments (e.g., by compressing at least one of the first side portion 204, the second side portion 205, and/or the third portion 206 inwardly) as it releases the pre-tensioning force. Such a configuration may be particularly beneficial for comminuted patella fractures.

The body 102 may include any number of channels 208, and the number of channels 208 is not particularly limited as long as the channels 208 enable a flexible geometry.

In some embodiments, at least one channel 208 extends through the edge of the body 102 such that the first side portion 204 and the second side portion 205 are not in contact (e.g., to enable the first side portion 204 and the second side portion may be stretched apart from one another).

In some embodiments, the body 102 may include at least a first channel positioned between the first side portion 204 and the third portion 206, a second channel positioned between the second side portion 205 and the third portion 206, and a third channel positioned between the first side portion 204 and the second side portion 205, wherein the third channel extends through the edge of the body 102 such that the first side portion 204 and the second side portion 205 are not in contact.

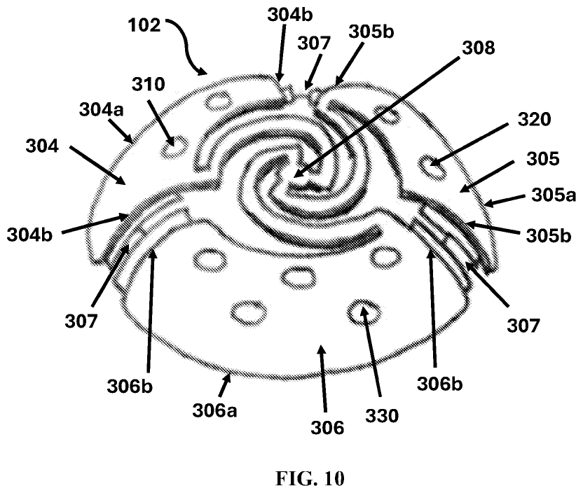

Referring to FIG. 10, a third exemplary embodiment of the body 102 configured to deliver multi-plane compression to the patella 16 may include a plurality of portions arranged annularly (e.g., positioned in a disk like manner). In some embodiments, a first portion 304, a second portion 305, and a third portion 306 may be arranged annularly, though the number of portions is not limited to such embodiment and any number of portions may be included. The first portion 304 may be configured to anchor to and/or engage a first portion of the anterior surface 16b of the patella 16. The second portion 305 may be configured to anchor to and/or engage a second portion of the anterior surface 16b of the patella 16. The third portion 306 may be configured to anchor to and/or engage a third portion of the anterior surface 16b of the patella 16.

The first portion 304 may be defined by one or more edges. For example, the first portion 304 may include one or more outer edges 304a and one or more side edges 304b. The edges may be any type of edge, such as straight, curved, sharp, rounded, and combinations thereof.

The second portion 305 may be defined by one or more edges. For example, the second portion 305 may include one or more outer edges 305a and one or more side edges 305b. The edges may be any type of edge, such as straight, curved, sharp, rounded, and combinations thereof.

The third portion 306 may be defined by one or more edges. For example, the third portion 306 may include one or more outer edges 306a and one or more side edges 306b. The edges may be any type of edge, such as straight, curved, sharp, rounded, and combinations thereof.

Any two of the first portion 304, the second portion 305, and the third portion 306 may be interconnected via a connecting joint 307. The connecting joint 307 may allow for the variable distances (e.g., extensions or flexions) between the first portion 304, the second portion 305, and/or the third portion 306 as the body 102 is stretched/pre-tensioned and released. For example, when the body 102 is an initial, non-tensioned state, there may be a first distance between two of the first portion 304, the second portion 305, and the third portion 306, and when the body 102 is stretched, the connecting joint 207 enables a second, larger distance between two of the first portion 304, the second portion 305, and the third portion 306.

In some embodiments, one or more side edges 304b of the first portion 304 may be connected to one or more side edges 305b of the second portion 305 via a connecting joint 307, and opposite side edges 304b of the first portion 304 may be connected to one or more side edges 306b of the third portion 306 via a connecting joint 307. Similarly, in some embodiments, one or more side edges 305b of the second portion 305 may be connected to one or more side edges 304b of the first portion 304 via a connecting joint 307, and opposite side edges 305b of the second portion 305 may be connected to one or more side edges 306b of the third portion 306 via a connecting joint 307. Similarly, in some embodiments, one or more side edges 306b of the third portion 306 may be connected to one or more side edges 304b of the first portion 304 via a connecting joint 307, and opposite side edges 306b of the third portion 306 may be connected to one or more side edges 305b of the second portion 305 via a connecting joint 307.

The first portion 304 may include a plurality of holes 310 extending through the body 102. The holes 310 can be configured to receive fasteners (not shown) to anchor the first portion 304 to the patella 16, and/or to receive wire, suture, cerclage wire, and combinations thereof (not shown) configured to compress the body 102. In some embodiments, the holes 310 may be spaced apart and arranged in a plurality of positions within the first portion 304. By providing spaced apart holes 310 in a plurality of positions, the body 102 may provide for flexibility in positioning the member 100 in relation to the patella 16. Further, providing spaced apart holes 310 in a plurality of positions may allow for anchoring of the first portion 304 to the patella via fasteners at different positions, which may help distribute loads and/or secure the entirety of the first portion 304 to the patella 16. Even further, providing spaced apart holes 310 in a plurality of positions may provide for flexibility in how a wire, suture, cerclage wire, and combinations thereof may be thread throughout the body 102.

The first portion 304 may include any number of holes 310. In some embodiments, the first portion 304 includes one hole, two holes, three holes, four holes, five holes, six holes, seven holes, eight holes, nine holes, ten holes, etc.

The first portion 304 may include one or more projections (not shown) extending from the one or more outer edges 304a. For example, the projections may be convex/rounded so as to conform with the shape of the anterior surface 16b of the patella 16.

The first portion 304 may include any number of projections. In some embodiments, the first portion 304 includes one projection, two projections, three projections, four projections, five projections, six projections, etc.

In some embodiments, at least one of the projections may include a hole 310 configured to receive a fastener to anchor the first portion 304 to the patella 16. In some embodiments, some or all of the projections may include a hole 310 configured to receive a fastener to anchor the first portion 304 to the patella 16.

The fasteners used in the first portion 304 may be screws, bolts, or any other suitable fastener and/or combination thereof.

The fasteners may be configured to as lateral fasteners (e.g., lateral screws) such that the fasteners extend into the anterior surface 16b of the patella 16 in or substantially in a perpendicular manner.

The fasteners may alternatively or additionally be configured as lag fasteners (e.g., lag screws) such that the fasteners extend into the anterior surface 16b of the patella 16 at an angle. Lag fasteners are typically configured to provide compression between two fragments of bone, and is therefore contemplated that the lag fasteners may extend into and/or through more than one bone fragment.

In some embodiments, at least one of the projections may be configured as a hook (not shown). The hook may be configured to fit around the patella 16, such that the first portion 304 grasps a side portion of the anterior surface 16b of the patella 16 to anchor the first portion 304 to the patella 16.

The first portion 304 may therefore be anchored to the patella 16 via fasteners, hooks, or a combination thereof. In preferred embodiments, the first portion 304 may be anchored to the patella without removing bone mass or disrupting soft tissue.

The second portion 305 may include a plurality of holes 320 extending through the body 102. The holes 320 can be configured to receive fasteners (not shown) to anchor the second portion 305 to the patella 16, and/or to receive wire, suture, cerclage wire, and combinations thereof (not shown) configured to compress the body 102. In some embodiments, the holes 320 may be spaced apart and arranged in a plurality of positions within the second portion 305. By providing spaced apart holes 320 in a plurality of positions, the body 102 may provide for flexibility in positioning the member 100 in relation to the patella 16. Further, providing spaced apart holes 320 in a plurality of positions may allow for anchoring of the second portion 305 to the patella via fasteners at different positions, which may help distribute loads and/or secure the entirety of the second portion 305 to the patella 16. Even further, providing spaced apart holes 320 in a plurality of positions may provide for flexibility in how a wire, suture, cerclage wire, and combinations thereof may be thread throughout the body 102.

The second portion 305 may include any number of holes 320. In some embodiments, the second portion 305 includes one hole, two holes, three holes, four holes, five holes, six holes, seven holes, eight holes, nine holes, ten holes, etc.

The second portion 305 may include one or more projections (not shown) extending from the one or more outer edges 305a. For example, the projections may be convex/rounded so as to conform with the shape of the anterior surface 16b of the patella 16.

The second portion 305 may include any number of projections. In some embodiments, the second portion 305 includes one projection, two projections, three projections, four projections, five projections, six projections, etc.

In some embodiments, at least one of the projections may include a hole 320 configured to receive a fastener to anchor the second portion 305 to the patella 16. In some embodiments, some or all of the projections may include a hole 320 configured to receive a fastener to anchor the second portion 305 to the patella 16.

The fasteners used in the second portion 305 may be screws, bolts, or any other suitable fastener and/or combination thereof.

The fasteners may be configured to as lateral fasteners (e.g., lateral screws) such that the fasteners extend into the anterior surface 16b of the patella 16 in or substantially in a perpendicular manner.

The fasteners may alternatively or additionally be configured as lag fasteners (e.g., lag screws) such that the fasteners extend into the anterior surface 16b of the patella 16 at an angle. Lag fasteners are typically configured to provide compression between two fragments of bone, and is therefore contemplated that the lag fasteners may extend into and/or through more than one bone fragment.

In some embodiments, at least one of the projections may be configured as a hook (not shown). The hook may be configured to fit around the patella 16, such that the second portion 305 grasps a side portion of the anterior surface 16b of the patella 16 to anchor the second portion 305 to the patella 16.

The second portion 305 may therefore be anchored to the patella 16 via fasteners, hooks, or a combination thereof. In preferred embodiments, the second portion 305 may be anchored to the patella without removing bone mass or disrupting soft tissue.

The third portion 306 may include a plurality of holes 330 extending through the body 102. The holes 330 can be configured to receive fasteners (not shown) to anchor the third portion 306 to the patella 16, and/or to receive wire, suture, cerclage wire, and combinations thereof (not shown) configured to compress the body 102. In some embodiments, the holes 330 may be spaced apart and arranged in a plurality of positions within the third portion 306. By providing spaced apart holes 330 in a plurality of positions, the body 102 may provide for flexibility in positioning the member 100 in relation to the patella 16. Further, providing spaced apart holes 330 in a plurality of positions may allow for anchoring of the third portion 306 to the patella via fasteners at different positions, which may help distribute loads and/or secure the entirety of the third portion 306 to the patella 16. Even further, providing spaced apart holes 330 in a plurality of positions may provide for flexibility in how a wire, suture, cerclage wire, and combinations thereof may be thread throughout the body 102.

The third portion 306 may include any number of holes 330. In some embodiments, the third portion 306 includes one hole, two holes, three holes, four holes, five holes, six holes, seven holes, eight holes, nine holes, ten holes, etc.

The third portion 306 may include one or more projections (not shown) extending from the one or more outer edges 306a. For example, the projections may be convex/rounded so as to conform with the shape of the anterior surface 16b of the patella 16.

The third portion 306 may include any number of projections. In some embodiments, the third portion 306 includes one projection, two projections, three projections, four projections, five projections, six projections, etc.

In some embodiments, at least one of the projections may include a hole 330 configured to receive a fastener to anchor the third portion 306 to the patella 16. In some embodiments, some or all of the projections may include a hole 330 configured to receive a fastener to anchor the third portion 306 to the patella 16.

The fasteners used in the third portion 306 may be screws, bolts, or any other suitable fastener and/or combination thereof.

The fasteners may be configured to as lateral fasteners (e.g., lateral screws) such that the fasteners extend into the anterior surface 16b of the patella 16 in or substantially in a perpendicular manner.

The fasteners may alternatively or additionally be configured as lag fasteners (e.g., lag screws) such that the fasteners extend into the anterior surface 16b of the patella 16 at an angle. Lag fasteners are typically configured to provide compression between two fragments of bone, and is therefore contemplated that the lag fasteners may extend into and/or through more than one bone fragment.

In some embodiments, at least one of the projections may be configured as a hook (not shown). The hook may be configured to fit around the patella 16, such that the third portion 306 grasps a side portion of the anterior surface 16b of the patella 16 to anchor the third portion 306 to the patella 16.

The third portion 306 may therefore be anchored to the patella 16 via fasteners, hooks, or a combination thereof. In preferred embodiments, the third portion 306 may be anchored to the patella without removing bone mass or disrupting soft tissue.

The body 102 further includes an intermediate portion 308 positioned within or between the first 304, second 305, and third 306 portions. The intermediate portion 308 may be configured to engage a central portion of the anterior surface 16b of the patella 16.

The intermediate portion 308 is stretchable such that it may be pre-tensioned and movable from an initial position to one or more stretched positions. The initial position refers to an equilibrium (e.g., non-tensioned) state in which the intermediate portion 308 is neither stretched nor flexed and experiences a net zero force. The one or more stretched positions refer to a position after application of various levels of stretching to the intermediate portion 308. The intermediate portion 308 may therefore be movable from its initial position to one or more stretched positions by pre-tensioning/stretching the intermediate portion 308. In particular, the intermediate portion 308 is configured to be resiliently movable from its initial position to one or more stretched positions, such that the intermediate portion 108 may return to its initial position after being stretched, thereby compressing the first 304, second 305, and third 306 portions towards one another. The intermediate portion 308 may further be configured to be resiliently biased to return to its initial position after being stretched.

The intermediate portion 308 may therefore enable the member 100 to be configured as a compliant mechanism. Due the stretchable nature of the intermediate portion 308, the member 100 may be pre-tensioned/stretched to fit around the patella 16, and the member 100 may then compress bone fragments (e.g., by radially compressing the first 304, second 305, and third 306 portions toward one another) as it releases the pre-tensioning force within the intermediate portion 308 to return to its initial position.

The intermediate portion 308 may be defined by a flexible topology positioned within or between the first 304, second 305, and third 306 portions. The flexible topology enables the intermediate portion 308 to be pre-tensioned/stretched from an initial position to one or more stretched positions as described above. The flexible topology may specifically be configured for multi-plane/radial motion such that the first 304, second 305, and third 306 portions are pulled towards one another. Such multi-plane/radial motion may be particularly beneficial for comminuted patella fractures.

In some embodiments, the flexible topology may include spiral flexures. The term “spiral flexures” refers to elements of a winding and/or continuous shape such that the elements may resemble a spiral or coil, thus allowing the elements to flex in multiple directions. The flexible topology may include any number of spiral flexures, such as one spiral flexure, two spiral flexures, three spiral flexures, four spiral flexures, five spiral flexures, six spiral flexures, etc., though the number of spiral flexures is not particularly limited as long as the flexures enable a flexible geometry. In some embodiments with more than one spiral flexure, the spiral flexures may expand from a central joint and connect to the first 304, second 305, and third 306 portions. For example, a first spiral flexure may extend from a central join and connect with the first portion 304, a second spiral flexure may extend from a central join and connect with the second portion 305, and a third spiral flexure may extend from a central join and connect with the first portion 306.

In some embodiments, the flexible topology may include lattice structures. A lattice structure may include a plurality of adjacently arranged, interconnected cells that form a grid-like pattern. In particular, the lattice structure may include a plurality of straight lines that connect at various points to create a network, and the connections often form geometric shapes (e.g., triangles, quadrilaterals, etc.), thus forming a grid-like pattern.

In some embodiments, the flexible topology may include ay topology-optimized geometries that facilitates and/or promotes multi-plane/radial compression between the first 304, second 305, and third 306 portions.

In some embodiments, the flexible topology includes a combination of two or more topologies described above.

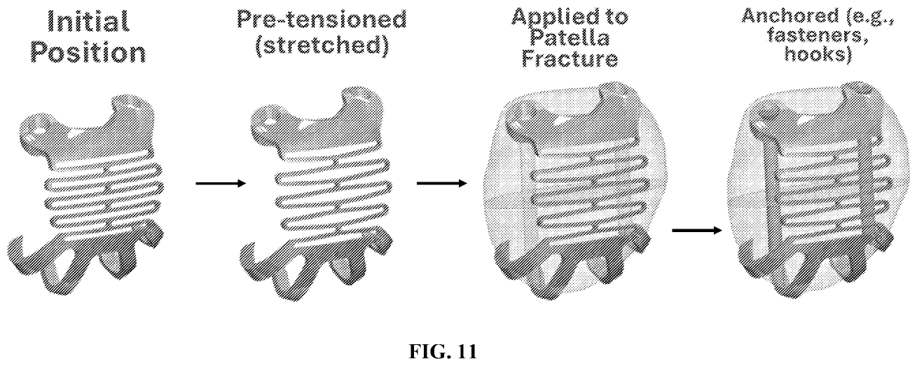

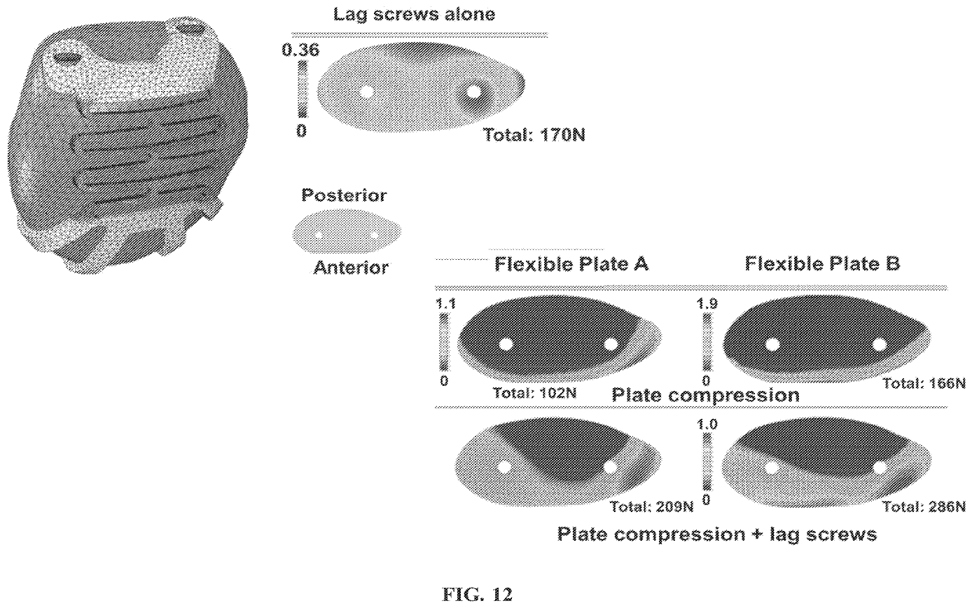

Embodiments further relate to methods of using the patella reinforcing member 100. Prior to using the member 100, it is understood that necessary steps may be required to adequately access the patella 16. For example, a patient's knee may be operated on, such as cut into, to expose and access the patella 16. Referring to FIG. 11, the member 100 may be pre-tensioned/stretched to fit around the patella 16. In some embodiments, the member 100 is stretched from its initial, non-tensioned position to one or more stretched states larger than the fractured patella. In preferred embodiments, the member 100 in its initial position is a smaller size than the fractured patella, thereby the member 100 must be stretched/pre-tensioned to fit around the fractured patella. While in its stretched state, the member 100 may be anchored to the patella 16 (e.g., via fasteners, hooks, etc.). The member 100 may then compress bone fragments as it releases its pre-tensioning force to return to its initial, non-tensioned position while anchored to the patella.

Embodiments further relate to a kit including a plurality of patella reinforcing members 100. In some embodiments, the kit may be provided with a plurality of members 100 of different dimensions. By providing members 100 with different dimensions, the kit can be provided to treat patients with different patella sizes. For example, a member 100 of a smaller size might be a better fit for a child or adolescent patient, while a member 100 of a larger size might be a better fit for an adult patient. In some embodiments, the kit may be provided with a plurality of members 100 of different configurations. By providing members 100 with different configurations, the kit can be provided to patients with different types of patella fractures. For example, at least a first member 100 of the kit may be configured to deliver single plane/linear compression, while at least a second member 100 of the kit may be configured to deliver multi-plane/radial compression.

The kit may further include fasteners, wire, suture, and/or cerclage wire.

The kit may further include one or more instruments, such as but not limited to forceps, drill guides, drill bits, and/or combinations thereof. In some embodiments, the kit may include a pre-tensioning insertion instrument configured to attach to the reinforcing member 100, expand the member 100 into its pre-tensioned state, and place the member 100 on a reduced patella fracture while fasteners are applied. The pre-tensioning instrument may be removed after the member 100 is adequately placed on the patella.

It should be understood that modifications to the embodiments disclosed herein can be made to meet a particular set of design criteria. For instance, the number of or configuration of components or parameters can be used to meet a particular objective.

It will be apparent to those skilled in the art that numerous modifications and variations of the described examples and embodiments are possible in light of the above teachings of the disclosure. The disclosed examples and embodiments are presented for purposes of illustration only. Other alternative embodiments can include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

It is the intent to cover all such modifications and alternative embodiments as can come within the true scope of this invention, which is to be given the full breadth thereof. Additionally, the disclosure of a range of values is a disclosure of every numerical value within that range, including the end points. Thus, while certain exemplary embodiments of the apparatus and process and/or utilization and methods of making and using the same have been discussed and illustrated herein, it is to be distinctly understood that the invention is not limited thereto but can be otherwise variously embodied and practiced within the scope of the following claims.

1-27. (canceled)

28. A patella reinforcing member, comprising:
a body having a first portion, a second portion, and a third portion arranged annularly;
an intermediate portion positioned between the first portion, the second portion, and the third portion, the intermediate portion sized and configured to be resiliently moveable from an initial position to a plurality of different stretched positions, the intermediate portion configured to be resiliently biased to return to the initial position when in any of the plurality of different stretched positions;
the first portion of the body being attachable to a first side of a patella bone, the second portion of the body being attachable to a second side of a patella bone, and the third portion of the body being attachable to a third side of a patella bone, such that when the body is in at least one of the plurality of different stretched positions when the first portion is attached to the first side of the patella bone, the second portion is attached to the second side of the patella bone, and the third portion is attached to the third side of the patella bone, the body exerts a radial compressive force on the patella bone.

29. The patella reinforcing member of claim 28, wherein the first portion of the body is attachable to the first side of the patella bone via one or more first fasteners, the second portion of the body is attachable to the second side of the patella bone via one or more second fasteners, and the third portion of the body is attachable to the third side of the patella bone via one or more third fasteners.

30. The patella reinforcing member of claim 28, wherein the intermediate portion is sized and configured to have a flexible geometry with one or more spiral flexures, the spiral flexures are each a first size when the intermediate portion is in the initial position and are each a second larger size when the intermediate portion is in one or more of the stretched positions.

31. The patella reinforcing member of claim 28, wherein the first portion of the body has one or more projections configured to hook onto the first side of the patella bone.

32. The patella reinforcing member of claim 28, wherein the first portion of the body has a plurality of spaced apart holes to receive first fasteners therein for attaching the first portion of the body to the first side of the patella bone.

33. The patella reinforcing member of claim 28, wherein the second portion of the body has one or more projections configured to hook onto the second side of the patella bone.

34. The patella reinforcing member of claim 28, wherein the second portion of the body has a plurality of spaced apart holes to receive second fasteners therein for attaching the second portion of the body to the second side of the patella bone.

35. The patella reinforcing member of claim 28, wherein the third portion of the body has one or more projections configured to hook onto the third side of the patella bone.

36. The patella reinforcing member of claim 28, wherein the third portion of the body has a plurality of spaced apart holes to receive third fasteners therein for attaching the third portion of the body to the distal side of the patella bone.

37. A method of attaching a patella reinforcing member onto a patella bone that is fractured, the method comprising:
positioning the patella reinforcing member of claim 28 onto the patella bone so that the body is in at least one of the plurality of different stretched positions while the first portion of the body is attached the first side of the patella bone, the second portion of the body is attached to the second side of the patella bone, and the third portion is attached to the third side of the patella bone such that the body exerts a radial compressive force on the patella bone.

38. The method of claim 37, comprising:
cutting into a knee of a patient to access the patella bone that is fractured.

39. The method of claim 38, comprising:
passing fasteners through the patella reinforcing member and the patella bone to attach the patella reinforcing member to the patella bone so that the body is in at least one of the plurality of different stretched positions while the first portion of the body is attached to the first side of the patella bone, the second portion of the body is attached to the second side of the patella bone, and the third portion is attached to the third side of the patella bone such that the body exerts a radial compressive force on the patella bone.
